(19) United States
(12) Patent Application Publication
(10) Pub. No.: US 2020/0225247 A1
(43) Pub. Date: Jul. 16, 2020

(54) METHODS AND KITS FOR PREDICTING THE TRANSPLANTATION-FREE SURVIVAL TIME OF PATIENTS SUFFERING FROM CIRRHOSIS

(71) Applicants: INSERM (INSTITUTE NATIONAL DE LA SANTE ET DE LA RECHERCHE MEDICALE), Paris (FR); UNIVERSITE PARIS DESCARTES, Paris (FR); ASSISTANCE PUBLIQUE-HOPITAUX DE PARIS (APHP), Paris (FR); UNIVERSITE PARIS DIDEROT-PARIS 7, Paris (FR)

(72) Inventors: Pierre-Emmanuel RAUTOU, Paris (FR); Chantal BOULANGER-ROBERT, Paris (FR)

(21) Appl. No.: 16/495,278

(22) PCT Filed: Mar. 20, 2018
(86) PCT No.: PCT/EP2018/057040

§ 371 Date: Sep. 18, 2019

(30) Foreign Application Priority Data
Mar. 21, 2017 (EP) 17305315.8

(51) Int. Cl. G01N 33/68; G01N 33/543

(52) U.S. Cl. CPC G01N 33/6893; A61K 45/06; G01N 2800/085; G01N 33/543

(57) ABSTRACT

Following a prospective clinical study that includes 242 patients, the inventors show that hepatocyte-derived MV levels predicted transplantation-free survival at 6 months in univariate analysis. In multivariate analysis, this association was shown to be independent of Child-Pugh and of MELD score. Thus the present invention thus relates to a method of predicting the transplantation-free survival time of a patient suffering from cirrhosis comprising determining the level of hepatocyte-derived microvesicles (e.g. by an ELISA assay) in a blood sample obtained from the patient.

### FIELD OF THE INVENTION

The present invention relates to methods and kits for predicting the transplantation-free survival time of patient suffering from cirrhosis.

### BACKGROUND OF THE INVENTION

Cirrhosis is a chronic disease of the liver. Cirrhosis prevalence will significantly increase during the next decade. Cirrhosis can result from a number of chronic liver diseases such as alcoholic liver disease, chronic viral hepatitis, non-alcoholic steatohepatitis, autoimmune diseases of the liver (primary biliary cirrhosis, primary sclerosing cholangitis, and autoimmune hepatitis). Cirrhosis progresses over several years. Mortality in cirrhosis is thus usually a consequence of decompensation or its ensuing complications. These complications include ascites (30 000 patients per year in France), gastrointestinal bleeding (10,000 episodes/year in France), renal failure and bacterial infections which is very common and often due to the translocation of Gram-negative intestinal bacteria. The treatment of choice for decompensated cirrhosis is liver transplantation and many such patients are placed on transplant waiting lists. Due to organ shortage, predicting the transplantation-free survival time of patients with cirrhosis is highly desirable to prioritize patients for transplantation. The MELD (Model for End Stage Liver Disease) score is currently used for organ allocation. Although the MELD score predicts 90-day mortality based on bilirubin, INR (international normalized ratio) and serum creatinine, this score is not a perfect prognostic tool and some cirrhotic patients may be misclassified and die on the waiting list for liver transplantation. MELD thus needs to be improved.

### SUMMARY OF THE INVENTION

The present invention relates to methods and kits for predicting the transplantation-free survival time of patient suffering from cirrhosis. In particular, the present invention is defined by the claims.

### DETAILED DESCRIPTION OF THE INVENTION

The first object of the present invention relates to a method of predicting the transplantation-free survival time of a patient suffering from cirrhosis comprising determining the level of hepatocyte-derived microvesicles in a blood sample obtained from the patient and ii) comparing the level determined at step i) with a predetermined reference value wherein a difference between the level determined at step i) and the predetermined reference value is indicative of the survival time of the patient.

As used herein, the term “cirrhosis” refers to a consequence of chronic liver disease characterized by replacement of liver tissue by fibrosis, scar tissue and regenerative nodules (lumps that occur as a result of a process in which damaged tissue is regenerated), leading to loss of liver function. Cirrhosis is most commonly caused by alcoholism, hepatitis B and C, and fatty liver disease (e.g. non-alcoholic steatohepatitis), but has many other possible causes.

As used herein, the expression “short transplantation-free survival time” indicates that the patient will have a transplantation-free survival time that will be lower than the median (or mean) observed in the general population of patient with cirrhosis. When the patient will have a short transplantation-free survival time, it is meant that the patient will have a “poor prognosis” and is at high risk of death or liver transplantation. Conversely, the expression “long transplantation-free survival time” indicates that the patient will have a transplantation free survival time that will be higher than the median (or mean) observed in the general population of patient with cirrhosis and that he/she may survive and not require liver transplantation. When the patient will have a long transplantation-free survival time, it is meant that the patient will have a “good prognosis”.

As used herein the term “blood sample” means a whole blood, serum, or plasma sample obtained from the patient. Preferably the blood sample, according to the invention, is a plasma sample. A plasma sample may be obtained using methods well known in the art. For example, blood may be drawn from the patient following standard venipuncture procedure on tri-sodium citrate buffer. Plasma may then be obtained from the blood sample following standard procedures including but not limited to, centrifuging the blood sample at about 2500*g for about 15 minutes (room temperature), followed by pipeting of the plasma layer. Platelet-free plasma (PFP) will be obtained following a second centrifugation at about 2500*g for 15 min. Analyses can be performed directly on this PFP. Alternatively, microvesicles (MVs) may be more specifically isolated by further centrifuging the PFP at about 15,000 to about 25,000*g at 4° C. Different buffers may be considered appropriate for resuspending the pelleted cellular debris which contains the MVs. Such buffers include reagent grade (distilled or deionized) water and phosphate buffered saline (PBS) pH 7.4. Preferably, PBS buffer (Sheath fluid) or NaCl 0.9% is used.

As used herein the term “microvesicle” or “MV” has its general meaning in the art and denotes a plasma membrane vesicle shed from an apoptotic or activated cell. The size of microvesicles ranges from 0.1 μm to 1 μm in diameter. The surface markers of microvesicles are the same as the cells from they originated. As sued herein the term “hepatocyte-derived microvesicle” refers to a microvesicle that derive from hepatocyte. Hepatocytes-derived microvesicles are characterized by the expression of cytokeratin-18. As used herein the term “cytokeratin-18” or “CK18” has its general meaning in the art and refers to the protein encodes by KRT18 gene (Gene ID 3875). An exemplary human amino acid sequence of cytokeratin-18 is represented by the NCBI reference sequence NP_000215.1.

Standard methods for determining the level of hepatocytes-derived microvesicles in a blood sample are well known in the art. For instance, circulating microvesicles can be isolated from the blood sample by centrifugation or by filtration on 0.2 um pore membranes and then contacting them with a set of binding partners directed against the specific surface markers of said microvesicles (Gastroenterology. 2012 July;143(1):166-76.e6.).

In some embodiments, the binding partner may be an antibody that may be polyclonal or monoclonal, preferably monoclonal, directed against the specific surface marker of microvesicles. Polyclonal antibodies of the invention or a fragment thereof can be raised according to known methods by administering the appropriate antigen or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies of the invention or a fragment thereof can be prepared and isolated using any technique that provides for the production of antibody molecules by continuous cell lines in culture. Techniques for production and isolation include but are not limited to the hybridoma technique; the human B-cell hybridoma technique; and the EBV-hybridoma technique.

In some embodiments, the binding partner is antibody which binds to M30 or M65 cytokeratin-18 fragment. As used herein, the term “M30 cytokeratin-18 fragment” refers to the caspase cleaved fragment of human keratin 18 protein (or “cytokeratin-18,” “CK-18,” “keratin-18,” “K18”) encoded by the KRT18 gene, and is a serum indicator of cellular apoptosis. The fragment is specifically recognized by M30 antibody which detects a neoepitope mapped to positions 387 to 396 of a 21-kDa fragment of CK18 (CK18Asp396 neoepitope) that is only revealed after caspase cleavage of the protein and is postulated as a selective biomarker of apoptotic cell death (Leers M, et al. (1999) “Immunocytochemical detection and mapping of a cytokeratin 18 neo-epitope exposed during early apoptosis.”. J Pathol. 187 (5): 567-72.). As used herein, the term “M65 cytokeratin-18 fragment” refers to the soluble human keratin 18 protein (or “cytokeratin-18,” “CK-18,” “keratin-18,” “K18”) encoded by the KRT18 gene, and is a serum indicator of cellular death. The fragment is specifically recognized by M65 antibody which detects a common epitope present in the full-length protein as well as the 21-kDa caspase cleaved fragment and is thus believed to measure, in addition to apoptosis, intact CK18 that is released from cells undergoing necrosis (Kramer G, Erdal H, Mertens H J, Nap M, Mauermann J, Steiner G, Marberger M, Biven K, Shoshan M C, Linder S. Differentiation between cell death modes using measurements of different soluble forms of extracellular cytokeratin 18. Cancer Res. 2004;64:1751-1756.).

In some embodiments, the binding partner of the invention is labelled with a detectable molecule or substance, such as a fluorescent molecule, a radioactive molecule or any others labels known in the art. Labels are known in the art that generally provide (either directly or indirectly) a signal. As used herein, the term “labelled”, with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer of the invention may be labelled with a radioactive molecule by any method known in the art. For example radioactive molecules include but are not limited radioactive atom for scintigraphic studies such as I¹²³, I¹²⁴, In¹¹¹, Re¹⁸⁶, Re¹⁸⁸. Preferably, the antibodies against the surface markers are already conjugated to a fluorophore (e.g. FITC-conjugated and/or PE-conjugated).

The aforementioned assays may involve the binding of the binding partners to a solid support. Solid supports which can be used in the practice of the invention include substrates such as nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like. The solid surfaces are preferably beads. Since microvesicles have a diameter of roughly 0.1 to 1 μm, the beads for use in the present invention should have a diameter larger than 1 um. Beads may be made of different materials, including but not limited to glass, plastic, polystyrene, and acrylic. In addition, the beads are preferably fluorescently labelled.

In some embodiments, an ELISA method is used, wherein the wells of a microtiter plate are coated with a set of antibodies which recognize said the microvesicle of interest. The blood sample is then added to the coated wells. After a period of incubation sufficient to allow the formation of antibody-antigen complexes, the plate(s) can be washed to remove unbound moieties and a detectably labelled secondary binding molecule is added. The secondary binding molecule is allowed to react with any captured sample marker protein, the plate washed and the presence of the secondary binding molecule detected using methods well known in the art. Specificity for microvesicles can be obtained by filtrating the plasma prior to performing ELISA.

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of expression levels in properly banked historical patient samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the expression level in a group of reference, one can use algorithmic analysis for the statistic treatment of the determined levels in samples to be tested, and thus obtain a classification standard having significance for sample classification. The full name of ROC curve is Receiver Operator Characteristic Curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

Typically, when the level of microvesicles is higher than the predetermined reference value, then it is concluded that the patient will have a short transplantation-free survival time. On the contrary, when the level of microvesicles is lower than the predetermined reference value, then it is concluded that the patient will have a long transplantation-free survival time. Practically, high statistical significance values (e.g. low P values) are generally obtained for a range of successive arbitrary quantification values, and not only for a single arbitrary quantification value. Thus, in some embodiments, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P value) are retained, so that a range of quantification values is provided. This range of quantification values includes a “cut-off” value as described above. For example, according to this specific embodiment of a “cut-off” value, the outcome can be determined by comparing the expression level with the range of values which are identified. In some embodiments, a cut-off value thus consists of a range of quantification values, e.g. centered on the quantification value for which the highest statistical significance value is found (e.g. generally the minimum p value which is found). For example, on a hypothetical scale of 1 to 10, if the ideal cut-off value (the value with the highest statistical significance) is 5, a suitable (exemplary) range may be from 4-6. For example, a patient may be assessed by comparing values obtained by determining the level of microvesicles, where values greater than 5 reveal a poor prognosis and values less than 5 reveal a good prognosis. In some embodiments, a patient may be assessed by comparing values obtained by measuring the level of microvesicles and comparing the values on a scale, where values above the range of 4-6 indicate a poor prognosis and values below the range of 4-6 indicate a good prognosis, with values falling within the range of 4-6 indicating an intermediate occurrence (or prognosis).

The result given by the method of the invention may be used as a guide in selecting a therapy or treatment regimen for the patient. For example, when the patient has been determined as having a poor prognosis he can be eligible for a preventive treatment (e.g. administration of the new antiapoptotics, vasoactive drugs, antifibrotic agents) or even for liver transplantation.

A further object of the invention relates to a kit for performing the method of the invention comprising means for determining the level of hepatocyte-derived microvesicles in a blood sample obtained from said patient. The kit may include filtration means (e.g. filters) and a set of antibodies as above described. In some embodiments, the antibody or set of antibodies are labelled as above described. The kit may also contain other suitably packaged reagents and materials needed for the particular detection protocol, including solid-phase matrices, if applicable, and standards. Typically, the kits described above will also comprise one or more other containers, containing for example, wash reagents, and/or other reagents capable of quantitatively detecting the presence of bound antibodies. Typically compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the blood sample, a container which contains the antibody(s) used in the assay, containers which contain wash reagents (such as phosphate buffered saline, Tris-buffers, and like), and containers which contain the detection reagent.

The invention will be further illustrated by the following figures and examples. However, these examples and figures should not be interpreted in any way as limiting the scope of the present invention.

### FIGURES

FIG. 1. Circulating hepatocyte MV levels (U/L) according to the Child-Pugh score. A) Test cohort. B) Validation cohort.

FIG. 2. Cumulative incidence of death according to circulating hepatocyte MV levels (U/L). A) Test cohort. B) Validation cohort.

FIG. 3. Cumulative incidence of death according to MELD and circulating hepatocyte MV levels (U/L). A) Test cohort. No death occurred in the group of patients with MELD>15 and hepatocyte MV≤65 U/L so that this group is not represented. B) Validation cohort. No death occurred in the group of patients with MELD≤15 and hepatocyte MV>65 U/L so that this group is not represented. HMV, hepatocyte microvesicle.

### EXAMPLE

### Material and Methods

### Patients

All consecutive patients with severe liver fibrosis or cirrhosis undergoing hepatic vein and/or right heart catheterization in two independent centers were prospectively included. The inclusion period ranged from June 2013 to March 2015 for the test cohort (Hôpital Beaujon, Clichy, France) and from January 2014 to December 2015 for the validation cohort (Hospital Clinic, Barcelona, Spain). Clinical, laboratory and hemodynamic features were prospectively collected in both centers. Diagnosis of severe liver fibrosis (METAVIR F3) or cirrhosis was based either on histological criteria or on the combination of clinical, laboratory, morphological and hemodynamic features. Non-inclusion criteria were a history of transjugular intrahepatic porto-systemic shunt, liver transplantation, hepatocellular carcinoma (HCC) outside Milan criteria since HCC increases MV levels by itself (Campello Thromb Res 2016; Brodsky J Gastrointestin Liver Dis 2008; Julich-Haertel, J Hepatol 2017), active extra-hepatic cancer, human immunodeficiency virus infection, primary sclerosing cholangitis and primary biliary cirrhosis since HVPG might not reflect portosystemic gradient in patients with cholestatic liver disease (Garcia-Tsao, Hepatology 2017), Budd-Chiari syndrome, or an acute event (hepato-renal syndrome, bacterial infection, alcoholic hepatitis, variceal bleeding) within two weeks prior to hepatic vein catheterization. Patients on the waiting list for liver transplantation and those with HCC were seen every 3 months. Other patients were seen every 6 months. When patients did not attend the follow-up visit, they were called by phone. In the absence of answer, we consulted the liver transplant registry and the national registry of deaths. This study was approved by the Institutional Review Boards of Paris North Hospitals, Paris 7 University, AP-HP (N° 11-112) and of Hospital Clinic (Barcelona, Spain). All patients included in this study gave written informed consent. The study conformed to the ethical guidelines of the 1975 Declaration of Helsinki.

### Platelet-Free Plasma Preparation

Platelet-free plasma was prepared at each centre following a standardized protocol proposed by Lacroix and colleagues. The principal investigator of this study trained the investigators responsible for plasma preparation to this procedure. Briefly, peripheral venous blood was collected within 2 hours prior to liver catheterization from the cubital vein of the patients, with a 21-gauge tourniquet needle, in 0.129 mol/L citrated tubes, after having discarded the first mL of blood. Tubes then remained motionless in the up-right position at room temperature for a maximum of 2 hours until platelet free plasma preparation, consisting in two successive centrifugations, each of 15 min at 2500 g at 20° C. with a light brake. Aliquots of platelet free plasma were then stored at −80° C. until use.

In some patients, hepatic venous blood was collected from the median or the right hepatic vein using a tip-curved catheter (Cook, HNB7.0-38-100-P-NS-MPA) and was prepared as mentioned above.

### Characterization of Circulating Microvesicle Levels

Circulating levels of annexin V+, platelet (CD41+), leuko-endothelial (CD31+/41−), pan-leukocyte (CD11a+) and endothelial (CD62e+ and CD144+) MVs were determined on a Gallios flow cytometer (Beckman Coulter, Villepinte, France) using a technique previously described. Regions corresponding to MVs were identified in forward light scatter and side-angle light scatter intensity dot plot representation set at logarithmic gain. MV gate was defined, using calibration beads (Megamix plus FSC, Biocytex, France), as events having a 0.1-1 μm diameter. This gate was separated into “large” (0.5 to 1 μm) and “small” (<0.5 μm) MVs. Events were then plotted on a fluorescence/forward light scatter dot plot to determine MV counts positively labeled by specific antibodies. Anti-CD41-Phycoerythrin-Cyanin7, anti-CD31-Phycoerythrin, anti-CD11a-Phycoerythrin, and anti-CD144-Phycoerythrin antibodies as well as their matched isotype controls were obtained from Beckman-Coulter. Anti-CD62E-fluoroisothiocyanate antibodies as well as their matched isotype controls were obtained from R&D Systems Europe. Annexin V fluoroisothiocyanate was purchased from Beckman-Coulter. MV concentration was assessed by comparison with a known amount of flowcount calibrator beads (AccuCount Fluorescent Particles, Spherotech, Chicago, 20 μL) added to each sample just before performing flow cytometry analysis. To limit variability, all measurements of MV levels by flow cytometry were performed using a unique batch for each antibody.

We determined plasma levels of hepatocyte-derived MVs using a technique previously described. Briefly, we measured circulating cytokeratin-18 levels (M65 EpiDeath ELISA, Peviva, Bromma, Sweden) before and after filtration of the plasma through two 0.2 μm filters (Ceveron MFU 500, Technoclone, Austria). The difference between soluble cytokeratin-18 levels in initial and in 0.2 μm filtrated platelet-free plasma reflected the concentration in hepatocyte MVs (data not shown). We also determined plasma levels of extracellular vesicles having a size ranging from 0.02 to 0.2 μm, corresponding to small MVs and/or exosomes, further referred to as “hepatocyte exosomes”. These levels corresponded to the difference between cytokeratin-18 levels in 0.2 μm filtrated and in 0.02 um filtrated (Whatman™ 6809-1002 Anotop™ Syringe Filter) platelet-free plasma. Hepatocyte MV and exosome levels were expressed as units per liter (U/L), according to the manufacturer's instructions. All ELISA were performed in duplicate with determination of the coefficient of variation between samples from the same patient. The results were considered adequate when the coefficient of variation was less than 20%. Otherwise, samples were measured again.

### C Reactive Protein and Interleukin 6 Concentrations Measurements

C reactive protein (DY1707, R&D Systems Europe, France), and Interleukin-6 (DY008, R&D Systems Europe, France) concentrations were measured in patients' plasma samples according to the manufacturer's instructions.

### Hemodynamic Evaluation

In both centers, HVPG was assessed using a technique previously described. The principal investigator of this study went to Barcelona to homogenize HVPG measurement between both centres. Briefly, after an overnight fasting, local anesthesia was performed and an introducer was placed under ultrasound guidance using the Seldinger technique. A 7 French balloon catheter (Lemaitre Vascular, for the test cohort; Edwards Lifesciences™, Irvine, Calif., USA for the validation cohort) was inflated in the right or median hepatic vein and wedge hepatic venous pressure was measured. Then, free hepatic venous pressure was obtained. HVPG was calculated as the difference between wedged and free hepatic venous pressures. Adequate occlusion was confirmed by injection of 5 mL of iodinated radiologic contrast medium. Permanent tracings were recorded. Clinically significant portal hypertension was defined as a HVPG≥10 mm Hg. When indicated, right heart hemodynamic measurements including pulmonary artery pressure, right atrial pressure, and pulmonary capillary wedge pressure were also performed using a Swan-Ganz catheter (Edwards Life Sciences). Cardiac index was measured by the thermodilution method and obtained by the average of 3 to 5 consecutive measurements.

### Histological Analysis

Liver tissue samples obtained from patients of the test cohort within 3 months before or after venous blood collection for MV measurement were retrospectively reviewed by an expert pathologist unaware of the results of MV measurements. The following features were analyzed on hematein and eosin, and on picrosirius stained tissue sections using semi-quantitative scoring defined a priori. Fibrosis was evaluated using picrosirius staining and scored according to Metavir Hepatology 1996;24:289-93; in case of cirrhosis (F4), the Laennec scoring system was used (stage 4a, mild/definitive cirrhosis with marked septation and visible nodules although most septa are thin; stage 4b, moderate cirrhosis with at least two broad septa and less than half of the tissue section composed of micronodules (<3 mm); stage 4c, severe cirrhosis with at least one very broad septum or more than half of the tissue section composed of micronodules) (J Hepatol 2011;55:1004-9). Activity was classified as absent to moderate vs. severe. Presence of apoptotic hepatocytes was also evaluated.

### Statistical Analyses

Quantitative variables were expressed as median (interquartile range) and categorical variables as frequencies. Comparisons of independent quantitative variables between groups were performed using the Mann-Whitney test. Comparisons of hepatocyte MV levels between hepatic and peripheral vein were performed using the Wilcoxon test. Spearman correlation analyses were used to evaluate the relationships between MV circulating levels, clinical and hemodynamic features. Follow-up time was defined as the period from the date of liver catheterization to 6 months after this procedure. Study outcome was evaluated using a multistate model as recommended in cirrhotic patients: data for patients who had not died were censored at the date of the last follow-up visit and were coded 0; data for patients who died before liver transplantation were coded 1; liver transplantation was considered to be a competing risk event and data were coded 2. A cumulative incidence function of death was calculated to describe the probability of death at a given time and was reported at 6 months with a 95% confidence interval. Univariate regression analyses were conducted using the Fine and Gray proportional hazards models to identify whether MV levels at baseline were associated with 6-month mortality. The value of MV level with the best sensitivity and specificity in area under the receiver operating characteristic curve analysis (Youden's Index) for death was chosen for further analyses. Each MV subpopulation achieving a P value <0.05 was introduced into a multivariable Fine and Gray proportional hazards model with Child-Pugh score or MELD to adjust our analyses for these severity scores and determine whether these MV levels had a prognostic value independently of these scores. All statistical tests were two-sided. P values <0.05 were considered to be statistically significant. Statistical analyses and figures were performed using the SPSS statistical package 16.0 software (SPSS Inc., Chicago, Ill., United States) and GraphPad Prism 5 software, respectively. Survival analyses were performed using SAS 9.4 statistical software.

### Results

### Patients' Characteristics in the Test Cohort

One hundred and thirty nine patients were included in the test cohort. Their characteristics are presented in Table 1. The main cause of liver disease was excessive alcohol consumption. Indications for hepatic vein, with or without right heart, catheterization were evaluation before liver transplantation in 70 (50%) patients or before liver surgery in 7 (5%) patients or assessment of the severity and/or the cause of liver disease using a liver biopsy in 62 (45%) patients. During the 6 months follow-up, 20 (14%) patients underwent liver transplantation and 9 (6%) patients died. Causes of death were HCC related in 2; liver related in 5, including variceal bleeding in 1, Klebsiella oxytoca pneumonia in 1, acute on chronic liver failure in 1, acute alcoholic hepatitis complicated with pneumonia in 1 and complicated refractory hepatic hydrothorax in 1; and unknown in 2.

### Circulating MVs Levels According to the Severity of the Liver Disease

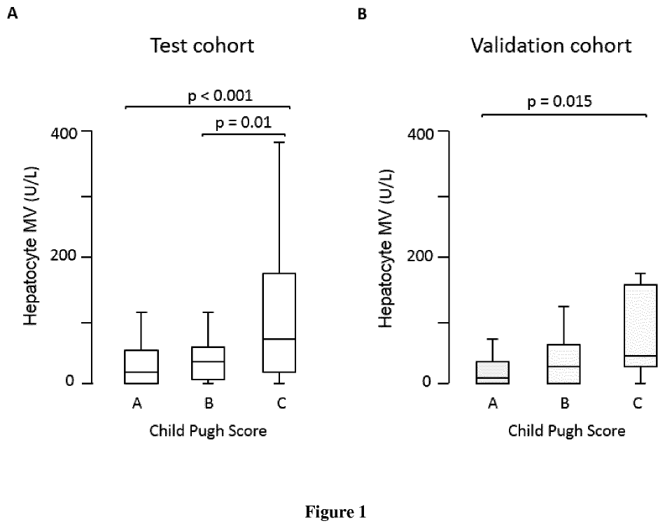

As shown in FIG. 1A (and data not shown), hepatocyte MV levels were 4.0 and 2.2 fold higher in patients with Child-Pugh C than in those with Child-Pugh A or B liver disease, respectively. Similar results were obtained when restricting the analysis to patients without HCC (data not shown). Hepatocyte MV levels weakly correlated with HVPG (r=0.22; p=0.011), and could not discriminate patients with HVPG >10 mm Hg from those having an HVPG below this threshold. Hepatocyte MV levels did not correlate with right heart hemodynamic values or with cardiac index, but correlated with markers of systemic inflammation (leukocytes, C-reactive protein, and interleukin 6) and with MELD and its components, and inversely correlated with serum sodium levels (data not shown). Severe liver necro-inflammatory activity and abundant liver fibrosis were associated with higher circulating levels of hepatocyte MVs (data not shown). There was also a trend towards higher hepatocyte MVs levels in patients with apoptotic hepatocytes. In ten additional patients (data not shown), we compared hepatocyte MV levels in hepatic vs. peripheral vein and observed 78% (27-233%, p=0.037) higher levels in hepatic than in peripheral vein from the same patients.

Total soluble cytokeratin 18 levels (bound and unbound to MVs) were slightly higher in patients with Child Pugh C than in those with Child Pugh B liver disease (data not shown for overall cohort and for patients without HCC), correlated weakly with HVPG (r=0.21; p=0.017), and did not significantly differ between patients with HVPG≥10 mm Hg and those with HVPG<10 mm Hg (data not shown).

Neither circulating levels of hepatocyte exosomes nor circulating levels of annexin V+, platelet (CD41+), leuko-endothelial (CD31+/41−), pan-leukocyte (CD11a+) and endothelial (CD62e+, CD144+) MVs measured by flow cytometry, were influenced by the severity of the liver disease, except for CD144+ MV levels found slightly higher in patients with Child-Pugh C liver disease in the overall cohort (data not shown) and CD31+/41− MV levels mildly higher in patients with Child-Pugh C liver disease without HCC (data not shown). Annexin V+, platelet, leuko-endothelial, pan-leukocyte and endothelial MV levels did not correlate with HVPG (data not shown) and could not identify patients with HVPG≥10 mm Hg (data not shown).

### Factors Associated with Six-Month Mortality

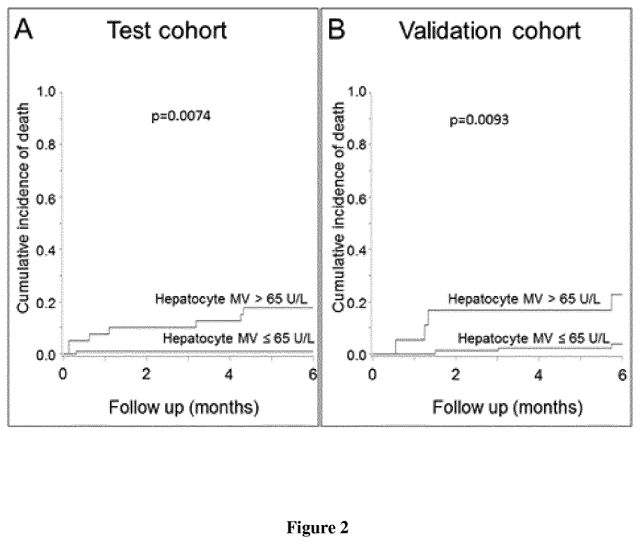

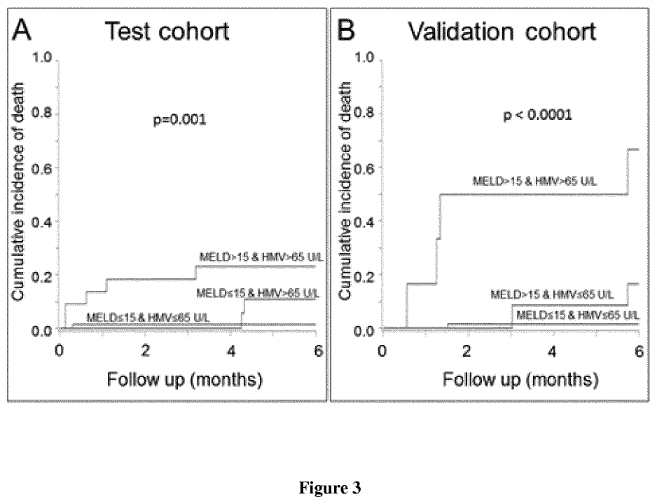

By univariate analysis, hepatocyte MV levels were strongly associated with 6-month mortality (Table 2). A cut-off value of 65 U/L yielded the most accurate sensitivity and specificity to identify patients' mortality. As shown in FIG. 2A, patients having hepatocyte MV levels>65 U/L had a 6-month cumulative incidence of death of 18% (8-31%) vs. 1% (1-5%) for patients having hepatocyte MV levels below this threshold. Hepatocyte MV level>65 U/L still predicted 6-month mortality after adjustment on Child-Pugh score or on MELD (Table 3). To further explore the added prognostic value of hepatocyte MV levels to MELD, we evaluated 6-month mortality according to the cut-off of 65 U/L and to MELD below or above 15, this threshold being recommended to list patients with end-stage liver disease for liver transplantation (1). As shown in FIG. 3A, patients with hepatocyte MV levels>65 U/L and MELD>15 were clearly at higher risk for 6-month mortality than the other patients (23% vs. 3%; p=0.001). Hepatocyte MV levels were also associated with 6-month liver-related mortality by univariate analysis (data not shown). Analyses adjusted on MELD and Child-Pugh score could not be performed since no liver-related death occurred in the group of patients with hepatocyte MV <65 in the test cohort.

We did similar prognostic analyses using total soluble cytokeratin 18 levels and found less obvious differences (Table 2 and data not shown). Hepatocyte MVs being a reflection of hepatocyte injury, we investigated the prognostic value of serum transaminase levels, but neither AST nor ALT levels were associated with 6-month mortality (data not shown). Other MV subpopulations did not predict mortality except for small CD³¹/41⁻MV levels that were associated with mortality by univariate (Table 2). When adjusting for severity scores of cirrhosis, CD³¹/41⁻ small MV levels predicted 6-month mortality independently of Child-Pugh score, but not of MELD (data not shown).

### Validation cohort

Characteristics of the 103 patients with cirrhosis included in the validation cohort are presented in Table 1. The main cause of liver disease was hepatitis C virus infection. Indications for hepatic vein, with or without right heart, catheterization were evaluation before liver transplantation in 4 (4%) patients or before liver surgery in 19 (18%) patients or assessment of the severity and/or the cause of liver disease by a liver biopsy in 80 (78%) patients. During the 6-month follow-up, 4 (4%) patients underwent liver transplantation, and 7 (7%) died. Causes of death were acute on chronic liver failure in 2 patients, septic shock in 1, variceal bleeding in 1, hemorrhagic stroke in 1 and HCC related in 2. The main results obtained in the test cohort were confirmed in the validation cohort, namely higher levels of hepatocyte MV levels in patients with Child-Pugh C liver disease (FIG. 1B; data not shown for the overall cohort and for patients without HCC), and a correlation with HVPG, but not with other hemodynamic values (data not shown). In the validation cohort, hepatocyte MVs levels were higher in patients with HVPG≥10 mm Hg than in those with an HVPG below this threshold [15 (0-63) vs. 5 (0-15); p=0.015]. Hepatocyte MV levels were associated with 6-month mortality by univariate analysis (Table 2, FIG. 2B) and after adjustment on Child-Pugh score (Table 3). Again, patients with hepatocyte MV levels>65 U/L and MELD>15 had 6-month mortality significantly higher than other groups (FIG. 3B). Hepatocyte MV levels were also associated with 6-month liver-related mortality by univariate analysis (data not shown). Analyses adjusted on MELD and Child-Pugh score were not performed due to the low number of events.

As CD144⁺ and CD31⁺/41⁻MV levels predicted patients mortality either in the test or in the validation cohort, we performed additional analyses to get further insight into the variables influencing circulating concentrations of these subpopulations of MVs in the 242 patients with cirrhosis. CD31⁺/41⁻ large MVs levels correlated with markers of systemic inflammation (C reactive protein and leukocytes) (data not shown).

TABLE 1
Baseline characteristics of the 242 patients with
advanced chronic liver disease

| | Test cohort | | Validation cohort | | |
|---|---|---|---|---|---|
| | (n = 139) | | (n = 103) | | p value |
| Clinical features | | | | | |
| Age (years) | 56 | (50-62) | 58 | (51-66) | 0.019 |
| Male gender-N (%) | 107 | (77) | 68 | (66) | 0.076 |
| Body Mass Index (kg/m²) | 26 | (23-30) | 26 | (23-29) | 0.985 |
| Fibrosis-N (%) | | | | | 0.005 |
| Advanced fibrosis (F3) | 10 | (7) | 0 | (0) | |
| Cirrhosis (F4) | 129 | (93) | 103 | (100) | |
| Cardiovascular risk factors-N (%) | | | | | |
| Hypertension | 50 | (36) | 23 | (22) | 0.022 |
| Smoking | 50 | (36) | 18 | (18) | 0.002 |
| Diabetes | 45 | (32) | 23 | (22) | 0.086 |
| Dyslipidemia | 13 | (9) | 8 | (8) | 0.665 |
| Causes of liver disease-N (%) | | | | | |
| Alcohol | 59 | (42) | 19 | (18) | <0.001 |
| Non alcoholic steatohepatitis | 37 | (27) | 3 | (3) | <0.001 |
| Hepatitis C | 41 | (29) | 77 | (75) | <0.001 |
| Hepatitis B | 10 | (7) | 4 | (4) | 0.2754 |
| Other | 16 | (12) | 2 | (2) | 0.005 |
| Ascites-N (%) | 76 | (55) | 23 | (22) | <0.001 |
| Hepatocellular carcinoma-N (%) | 43 | (31) | 20 | (19) | 0.044 |
| Child Pugh Class A/B/C | 43 (31)/52 (37)/ | | 66 (64)/29 (28)/ | | <0.001 |
| | 44 (32) | | 8 (8) | | |
| Model for end-stage liver disease (MELD) | 13 | (9-17) | 11 | (8-14) | 0.004 |
| Large varices esophageal or history of | 26 | (23) | 4 | (5) | |
| band ligation-N (%) | | | | | <0.001 |
| Laboratory data | | | | | |
| Serum sodium (mmol/L) | 136 | (134-138) | 141 | (138-143) | <0.001 |
| Serum creatinine (μmol/L) | 70 | (63-86) | 68 | (53-81) | 0.015 |
| Serum aspartate aminotransferase (ULN) | 1.81 | (1.3-2.8) | 1.63 | (1-2.75) | 0.465 |
| Serum alanine aminotransferase (ULN) | 1.1 | (0.6-1.98) | 1.3 | (0.8-2.15) | 0.143 |
| Serum bilirubin (μmol/L) | 33 | (14-64) | 24 | (14-43) | 0.039 |
| Leukocytes (10⁹/L) | 5.1 | (3.7-7.1) | 4.4 | (3.3-6) | 0.011 |
| Hemoglobin (g/dL) | 12.0 | (10.5-14.0) | 13.2 | (12.0-14.8) | 0.014 |
| Platelet count (G/L) | 93 | (68-138) | 85 | (59-134) | 0.271 |
| C Reactive protein (mq/L) | 4.5 | (1.6-8.8) | 1.8 | (0.4-4.1) | <0.001 |
| Interleukin 6 (pg/mL) | 9 | (0-27) | 0 | (0-24) | 0.108 |
| Hemodynamic data | | | | | |
| Hepatic venous pressure gradient (mm Hg) | 16 | (12-20) | 16 | (13-19) | 0.100 |
| ≥10 mm Hg-N (%) | 114 | (85) | 83 | (81) | |
| Wedge hepatic venous pressure (mm HQ) | 24 | (18-29) | 23 | (18-27) | 0.231 |
| Free hepatic venous pressure (mm Hq) | 7 | (5-10) | 8 | (6-9) | 0.664 |
| Heart rate (bpm) | 72 | (65-84) | 63 | (55-75) | <0.001 |
| Mean arterial pressure (mm Hg) | 91 | (85-102) | 90 | (80-98) | 0.012 |
| Right atrial pressure (mm Hg) | 4 | (2-5) | 5 | (3.5-6.5) | <0.001 |
| Mean pulmonary artery pressure (mm Hg) | 13 | (11-17) | 15 | (12-19) | 0.009 |
| Cardiac index (L/min/m²) | 3.5 | (2.8-4.4) | 3.4 | (2.8-4.5) | 0.964 |
| Beta-blockers treatment-N (%) | 69 | (50) | 45 | (44) | 0.359 |

Data are expressed as median (interquartile range) or number (%) as appropriate.

Some patients had several causes of cirrhosis.

Esophageal varices data were available in 111 patients in the test cohort and in 83 patients in the validation cohort. Hepatic venous pressure gradient, heart rate, mean arterial pressure, right atrial pressure, mean pulmonary artery pressure and cardiac index were available in the test cohort for 134, 139, 139, 134, 125, 125 patients respectively and in the validation cohort for 103, 101, 102,102, 47, 46 patients, respectively.

Abbreviations: bpm, beat per minute; ULN, upper limit of normal values.

TABLE 2
Univariate analyses evaluating the association of circulating microvesicle
(MV) subpopulation and soluble cytokeratin 18 levels with 6-month transplantation-free
survival using Gray's test (transplantation counted as competing risk, death counted as event).

| | Test cohort | | | Validation cohort | | |
|---|---|---|---|---|---|---|
| | Hazard | 95% confidence | | Hazard | 95% confidence | |
| Variable | ratio | interval | P value | ratio | interval | P value |
| Univariate analysis | | | | | | |
| Annexin V ⁺ 10³ | 0.877 | 0.748-1.029 | 0.108 | 1.049 | 0.977-1.127 | 0.190 |
| MVs/μL | | | | | | |
| Annexin V⁺ | 0.831 | 0.656-1.053 | 0.125 | 1.068 | 0.985-1.159 | 0.112 |
| small 10³ | | | | | | |
| MVs/μL | | | | | | |
| Annexin V⁺ large | 0.751 | 0.442-1.274 | 0.289 | 1.134 | 0.674-1.909 | 0.636 |
| 10³ MVs/μL | | | | | | |
| CD11a⁺ 10³ | 0.787 | 0.551-1.124 | 0.188 | 0.189 | 0.004-9.225 | 0.401 |
| MVs/μL | | | | | | |
| CD11a⁺ small | 0.704 | 0.326-1.521 | 0.798 | 0.064 | 0.000-18.559 | 0.342 |
| 10² MVs/μL | | | | | | |
| CD11a⁺ large | 0.611 | 0.288-1.298 | 0.200 | 0.031 | 0.000-985.24 | 0.512 |
| 10² MVs/μL | | | | | | |
| CD144⁺ 10² | 0.667 | 0.156-2.855 | 0.585 | 5.060 | 2.326-10.838 | <0.001 |
| MVs/μL | | | | | | |
| CD144⁺ small | 0.592 | 0.027-12.890 | 0.739 | 23.358 | 1.425-382.801 | 0.027 |
| 10² MVs/μL | | | | | | |
| CD144⁺ large | 0.470 | 0.052-4.231 | 0.501 | 9.238 | 4.566-18.69 | <0.001 |
| 10² MVs/μL | | | | | | |
| CD62E⁺ 10³ | 1.029 | 0.731-1.449 | 0.868 | 0.337 | 0.071-1.591 | 0.170 |
| MVs/μL | | | | | | |
| CD62E⁺ small | 1.047 | 0.638-1.720 | 0.855 | 0.186 | 0.006-5.369 | 0.327 |
| 10³ MVs/μL | | | | | | |
| CD62E⁺ large | 1.043 | 0.361-3.014 | 0.938 | 0.109 | 0.007-1.800 | 0.121 |
| 10³ MVs/μL | | | | | | |
| CD41⁺ 10³ | 0.966 | 0.795-1.174 | 0.729 | 1.036 | 0.698-1.538 | 0.861 |
| MVs/μL | | | | | | |
| CD41⁺ small 10³ | 0.871 | 0.623-1.219 | 0.421 | 0.972 | 0.475-1.991 | 0.939 |
| MVs/μL | | | | | | |
| CD41⁺ large 10³ | 1.042 | 0.611-1.776 | 0.881 | 1.386 | 0.503-3.818 | 0.528 |
| MVs/μL | | | | | | |
| CD31⁺/41⁻ 10³ | 0.086 | 0.003-2.678 | 0.1622 | 7.434 | 0.952-58.018 | 0.056 |
| MVs/μL | | | | | | |
| CD31⁺/41⁻ small | 0.010 | 0.000-0.732 | 0.036 | 20.211 | 0.254-1606.147 | 0.178 |
| 10³ MVs/μL | | | | | | |
| CD31⁺/41⁻ large | 0.024 | 0.000-44.352 | 0.331 | 61.075 | 1.500-2486.37 | 0.030 |
| 10³ MVs/μL | | | | | | |
| Hepatocyte | 1.000 | 0.997-1.002 | 0.735 | Non available | | |
| exosomes (U/L) | | | | | | |
| Hepatocyte MVs | 1.998 | 1.543-2.588 | <0.001 | 17.440 | 5.862-51.884 | <0.001 |
| 10³ (U/L) | | | | | | |
| Hepatocyte MVs | 17.560 | 2.157-143 | 0.0074 | 6.997 | 1.614-30.335 | 0.0093 |
| (U/L) | | | | | | |
| >65 vs. ≤65 U/L * | | | | | | |
| Total soluble | 1.544 | 1.353-1.761 | <0.001 | 1.544 | 1.330-1.792 | <0.001 |
| cytokeratine 18⁺ | | | | | | |
| 10³ (U/L) | | | | | | |
| Total soluble | 11.457 | 1.405-93.412 | 0.0227 | 4.299 | 0.986-18.744 | 0.0522 |
| cytokeratine 18⁺ | | | | | | |
| (U/L) >300 | | | | | | |
| vs ≤300 ** | | | | | | |
| Child-Pugh | 1.451 | 0.918-2.292 | 0.1108 | 2.054 | 1.566-2.694 | <0.0001 |
| score | | | | | | |
| MELD | 1.140 | 1.019-1.274 | 0.0216 | 1.635 | 1.376-1.943 | <0.0001 |

* 65 U/L: cut-point found with Youden index (with hepatic transplantation considered as censored);
** 300 U/L: cut-point found with Youden index (with hepatic transplantation considered as censored)
Bold indicates significant associations with survival.

TABLE 3
Analyses of the ability of hepatocyte MV levels to predict 6-month mortality
adjusted on Child Pugh Score and MELD (Gray's test, transplantation counted as competing
risk, death counted as event).

| | Test cohort | | | Validation cohort | | |
|---|---|---|---|---|---|---|
| | | 95% | | | 95% | |
| | Hazard | confidence | | Hazard | confidence | |
| Variable | ratio | interval | P value | ratio | interval | P value |
| Hepatocyte MVs (U/L) | 12.616 | 1.922-82.810 | 0.0083 | 4.892 | 1.283-18.662 | 0.0201 |
| >65 vs ≤65 | | | | | | |
| Child Pugh Score | 1.244 | 0.852-1.818 | 0.2583 | 2.009 | 1.472-2.740 | <0.0001 |
| Hepatocyte MVs (U/L) | 12.229 | 1.691-88.428 | 0.0131 | 3.484 | 0.972-12.482 | 0.0502 |
| >65 vs ≤65 | | | | | | |
| MELD | 1.087 | 0.978-1.208 | 0.1233 | 1.604 | 1.341-1.919 | <0.0001 |

Bold indicates significant associations with survival.

### REFERENCES

Throughout this application, various references describe the state of the art to which this invention pertains. The disclosures of these references are hereby incorporated by reference into the present disclosure.

1. A method of predicting the transplantation-free survival time of a patient suffering from cirrhosis comprising
determining the level of hepatocyte-derived microvesicles in a blood sample obtained from the patient, wherein when the level of microvesicles is higher than a predetermined reference value, then the patient will have a short transplantation-free survival time or when the level of microvesicles is lower than the predetermined reference value, then the patient will have a long transplantation-free survival time.

2. The method of claim 1 wherein the level of hepatocytes-derived microvesicles is determined by isolating microvesicles from the blood sample by centrifugation and then contacting the microvesicles with at least one binding partner directed against the specific surface markers of said hepatocytes-derived microvesicles.

3. The method of claim 2 wherein the at least one binding partner is a monoclonal antibody.

4. The method of claim 3 wherein the monoclonal antibody binds to M30 or M65 cytokeratin-18 fragment.

5. The method of claim 2 wherein the level of hepatocytes-derived microvesicles is determined by an ELISA assay.

6-7. (canceled)

8. A method of predicting the transplantation-free survival time of a patient suffering from cirrhosis and treating a patient with a short predicted transplantation-free survival time comprising
i) determining the level of hepatocyte-derived microvesicles in a blood sample obtained from the patient, wherein when the level of microvesicles is higher than a predetermined reference value, then the patient will have a short transplantation-free survival time or when the level of microvesicles is lower than the predetermined reference value, then the patient will have a long transplantation-free survival time, and
ii) administering a preventive treatment or transplanting a liver into a patient whose measurement is indicative of a short transplantation-free survival time.

9. The method of claim 8, wherein the preventive treatment includes administration of one or more of an antiapoptotic agent, a vasoactive drug and an antifibrotic agent.
